(19) United States
(12) Patent Application Publication
(10) Pub. No.: US 2011/0208158 A1
(43) Pub. Date: Aug. 25, 2011

(54) Apparatus and Method for Endoscopic Submucosal Dissection

(75) Inventors: John C. Sigmon, JR., High Point, NC (US); Vihar C. Surti, Winston-Salem, NC (US); Michael L. Williams, Clemmons, NC (US)

(73) Assignee: Cook Medical Technologies LLC, Bloomington, IN (US)

(21) Appl. No.: 13/029,295

(22) Filed: Feb. 17, 2011

Related U.S. Application Data
(60) Provisional application No. 61/306,100, filed on Feb. 19, 2010.

(51) Int. Cl. A61M 5/315; A61M 5/30

(52) U.S. Cl. 604/506; 604/222; 604/199

(57) ABSTRACT

A kit and a method for delivering an injectable solution to a tissue treatment site are provided. The kit includes a housing having a chamber, a proximal portion and a distal portion. An injectable solution having a viscosity greater than about 10,000 cP is provided in the chamber. A plunger is provided in the proximal portion of the housing. The kit also includes a pressure gauge operably connected to the housing. The kit may also include a handle connected to the housing and a plunger advancing member having a plunger handle connected thereto provided separate from the housing. The kit may also include an inner shaft provided separate from the housing and having a proximal end portion configured for operably connecting with the distal portion of the housing for receiving the injectable solution therethrough and a distal end of the shaft configured for insertion in to the tissue treatment site.

This application claims the benefit of U.S. Provisional Application No. 61/306,100, filed Feb. 19, 2010, which is incorporated by reference herein in its entirety.

### TECHNICAL FIELD

This invention generally relates to medical devices and in particular to an apparatus, kit and method for endoscopic submucosal dissection using an injectable solution.

### BACKGROUND

Minimally invasive medical procedures are performed in various passageways in the body using elongated instruments inserted through natural orifices or small surgical openings. In some procedures, it is desirable to treat some diseases using en bloc tissue removal through an elongate device, for example removing tissue lesions or polyps.

In some procedures, such as endoscopic submucosal dissection (ESD), a solution may be submucosally injected between layers of tissue to create a tissue elevation for removing the diseased tissue. Previously, injections of solutions such as saline or hyaluronic acid (HA) have been used to form an elevated tissue lesion for surgical removal. The elevated tissue is resected using a needle knife to cauterize the tissue or a snare to remove the elevated tissue section.

However, problems with the tissue resection occur when using solutions having a low viscosity such as saline. For example, the injected saline leaks out from between the tissue layers through the injection site, resulting in dissipation of the fluid, even when using multiple injection sites. Fluid dissipation leads to loss of leverage for removal of the tissue leading to risk of perforation of the underlying tissue and excessive bleeding. In addition, the viscosity of the saline solution is insufficient to cause enough pressure between the layers of the tissue to physically separate the layers to facilitate the removal of the diseased tissue. While solutions including HA are more viscous, HA solutions are expensive and not readily available in most endoscopy procedure suites. In addition, HA is hydrophilic, but requires dilution pre-injection that can lead to inconsistency with each injection.

There is a need for an apparatus and a method to provide an injectable solution for injection between tissue layers to form a tissue elevation and having sufficient pressure to physically break the cellular connections between the layers of healthy tissue and diseased tissue and to remain at the injection site for a sufficient time. In addition an apparatus and a method are needed to deliver an injectable solution to provide a tissue elevation that is present for a sufficient amount of time for a surgical procedure. There is also a need for a kit including an injectable solution having a consistent viscosity solution and a delivery apparatus for the injectable solution.

### SUMMARY OF THE INVENTION

Accordingly, it is an object of the present invention to provide a kit and a method having features that resolve or improve on one or more of the above-described drawbacks.

The foregoing object is obtained in one aspect of the present invention by providing a kit for delivering an injectable solution to a tissue treatment site. The kit includes a housing having a chamber, a proximal portion and a distal portion. An injectable solution having a viscosity greater than about 10,000 cP is provided in the chamber. The kit also includes a plunger movably positionable within the proximal portion of the chamber, the plunger provides a seal at the proximal end portion. In some embodiments, a pressure gauge is also provided with the kit. A handle is connected to the housing and a plunger advancing member having a plunger handle is connected thereto. In some embodiments, the plunger advancing member is provided separate from the housing and includes a distal portion configured for operably connecting with the proximal portion of the housing. The kit also includes an inner shaft provided separate from the housing and having a proximal end portion configured for operably connecting with the distal portion of the housing for receiving the injectable solution therethrough and a distal end configured for insertion in to the tissue treatment site.

In another aspect of the present invention, a kit is provided. The kit includes a housing having a chamber, a proximal portion and a distal portion. An injectable solution having a viscosity greater than about 10,000 cP is provided in the chamber. The kit also includes a plunger movably position within the proximal portion of the chamber, the plunger provides a seal at the proximal end portion. A plunger advancing member handle connected to the housing having a plunger handle connected thereto is also provided separate from the housing in the kit. The kit also includes an inner shaft provided separate from the housing and having a proximal end portion configured for operably connecting with the distal portion of the housing for receiving the injectable solution therethrough and a distal end configured for insertion in to the tissue treatment site.

In another aspect of the present invention, a method of elevating a first tissue layer away from a second tissue layer is provided. The method includes connecting an inner shaft to a distal portion of a housing having a chamber therein, connecting a plunger to a proximal portion of the housing and advancing a distal end of the inner shaft to the first tissue layer and inserting the distal end into the first tissue layer. The method also includes distally advancing the plunger to advance an injectable solution having a viscosity greater than about 10,000 cP from the chamber through the inner shaft and into the tissue and injecting the solution into the first layer and elevating the first tissue layer away from the second tissue layer.

### BRIEF DESCRIPTION OF THE DRAWINGS

FIG. 1 is a partial side view of a distal end of an embodiment of a delivery device at a tissue treatment site;

FIG. 2 is a partial side view of an embodiment of a delivery device in accordance with the present invention;

FIG. 3 is a partial side view of the embodiment shown in FIG. 2 in a first position;

FIG. 4 is a partial side view of the embodiment shown in FIG. 2 in a second position;

FIG. 5 is a side view of a proximal portion an embodiment of a the delivery device in accordance with the present in;

FIG. 6 is a side view of the embodiment of the delivery device shown in FIG. 5;

FIG. 7 illustrate an embodiment of a delivery device in accordance with an embodiment of the present invention;

FIGS. 8A and 8B are side views of adaptors and a tube of the delivery device shown in FIG. 7;

FIG. 9 illustrates an embodiment of a kit in accordance with the present invention;

FIG. 10 illustrates an embodiment of a kit in accordance with the present invention;

FIGS. 11A and 11B illustrate an alternative embodiment of a kit in accordance with the present invention; and

FIG. 12 illustrates an injectable solution being delivered to a treatment side with a delivery device in accordance with the present invention.

### DETAILED DESCRIPTION OF THE EMBODIMENTS

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician delivering the injectable solution to a patient. Hence the term “distal” means the portion of the device that is farthest from the physician and the term “proximal” means the portion of the device that is nearest to the physician.

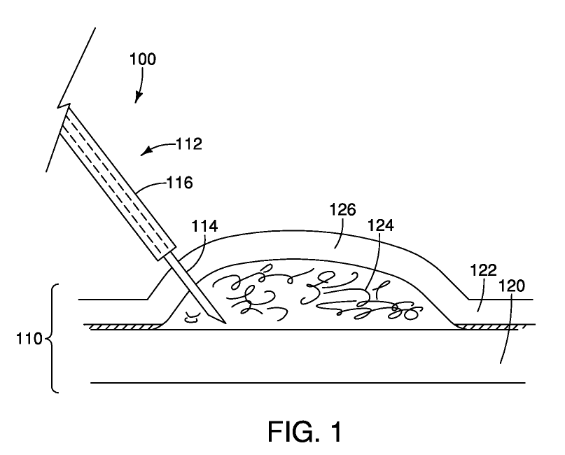

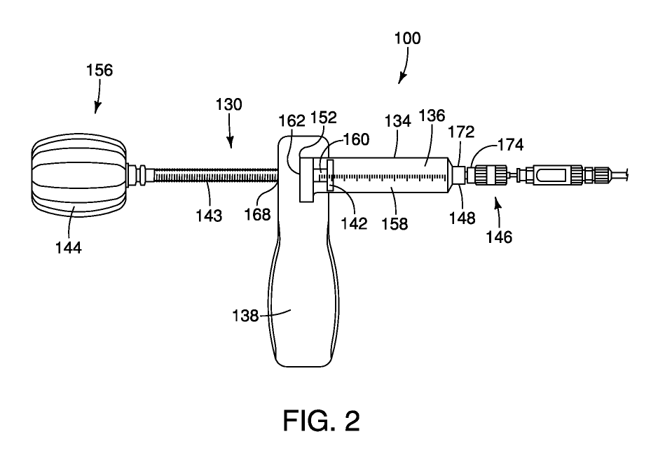

FIGS. 1 and 2 illustrate a delivery device 100 for delivering an injectable solution to a tissue treatment site 110. A distal portion 112 of the delivery device 100 is shown in FIG. 1 including an inner shaft 114 extending out of an outer catheter 116 so that the inner shaft 114 extends into the tissue 110. The inner shaft 114 may be a needle, cannula or other elongate tubular structure suitable for insertion into the tissue 110. The inner shaft 114 is inserted between a first layer of tissue 120 and a second layer of tissue 122. The layers 120, 122 may be any adjacent layers of tissue, for example, the muscularis and submucosal layers. As shown in FIG. 1, the injection of the solution between the first layer 120 and the second layer 122 forms a fluid-filled pocket 124 that forces separation between the first and second layers 120, 122, breaking the attachments between the tissue layers 120, 122. The elevated tissue portion 126 may then be resected by the physician using an electrocautery device or snare as described in more detail below.

A proximal portion 130 of the delivery device 100 is shown in FIG. 2. The proximal portion 130 includes a housing 134 having a chamber 136 formed therein. The device 100 further includes an injector handle 138 connected to the housing 134, a plunger 142 positioned within the housing 134, a plunger advancer member 143 and a plunger handle 144 operably connected to the plunger advancer member 143. The plunger advancer member 143 may be connected to the plunger 142 when the solution is ready to be delivered to the treatment site. A connector 146 is connected to a distal end portion 148 of the housing 134. The connector 146 removably connects the inner shaft 114 and the outer catheter 116 to the distal end portion 148 of the housing 134.

The plunger advancer member 143 is insertable into a proximal opening 152 of the housing 134 and fits on a portion of the plunger 142. The plunger 142 is advanceable toward the distal end portion 148 of the housing 134 to decrease the volume of the chamber 136 and advance the injectable solution into the tissue 110. In some embodiments, the plunger advancer member 143 is a screw-gear plunger having the plunger handle 144 at a proximal end 156 of the delivery device 100 and a distal end 158 received by the plunger 142 within the chamber 136 of the housing 134. The screw gear plunger member may include male or female threads or grooves that may be used to distally advance the plunger 142 to create pressure within the chamber 136 to force the injectable solution distally into the tissue 110. In some embodiment the plunger 142 may form a seal at the proximal end 156 so that the solution does not escape the proximal end 156. A seal (not shown) may be provided at the distal end 158 of the plunger 142 that seals the chamber 136 as the plunger 142 is distally advanced and prevents the injectable solution from flowing proximally past the plunger 142. The seal allows high pressure within the chamber 134 to distally advance the injectable solution through the inner shaft 114 without leaking. By way of non-limiting example, the seal may be an o-ring. In some embodiments, the seal may be provided in the form of a polytetrafluoroethylene (PTFE) tape. The PTFE tape may be wound around the end of the plunger 142 to form the seal between the plunger 142 and the housing wall 134. A distal seal may also be provided on the distal end of the housing 134 to seal the housing 134 for delivery and before the housing 134 is connected to a pressure gauge or a connector as discussed below.

The housing 134 may be adapted for withstanding positive displacement pressures associated with advancing injectable solutions having increased viscosity through the distal end 148 of the housing 134 and into the inner shaft 114. For example, the viscosity of the solution within the chamber 136 may be greater than about 10,000 cP. The housing 134 may be formed from any suitable material sufficient to withstand the pressure generated for a solution having a viscosity greater than about 10,000 cP. In some embodiments, the housing may accommodate a solution having a viscosity greater than about 30,000 cP. Materials for forming the housing may include, but are not limited to plastic, such as polycarbonate, and glass.

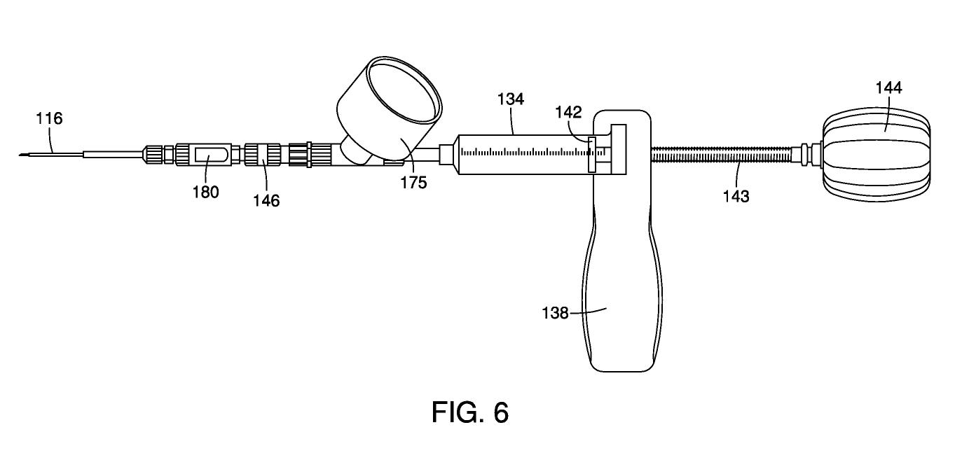

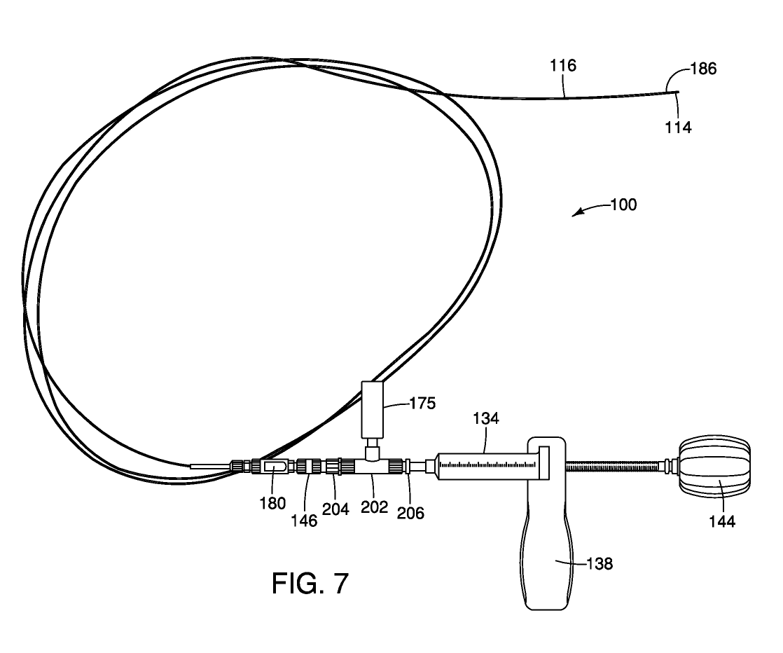

In some embodiments, the delivery device 100 includes a pressure gauge 175 as shown in FIG. 5. The pressure gauge is operably connected to the distal end 148 of the housing 134, for example, using a y-adaptor 177 having a connector 179 for connecting to the connector 146 that removably connects the inner shaft 114 and the outer catheter 116. The y-adaptor 177 may be glued to the connector 179 and to a connector 181 that connects to the distal end 148 of the housing 134 so that the high pressure solution does not leak from the connections. FIG. 6 illustrates the pressure gauge 175 operably connected to the housing 134 and the connector 146 connected to the outer catheter 116. The solution may be delivered through the inner shaft at a nominal pressure between about 1 psi to about 3000 psi. The delivery device 100 may also include an automatic stop that prevents the physician from exceeding a predetermined pressure, for example, if the inner catheter 114 becomes clogged or bent. One stop may be provided when the pressure exceeds about 2000 psi. In some embodiments, a stop may be provided with the pressure exceeds about 3000 psi. Other pressure cut offs may also be used. The amount of pressure measured will vary depending on the concentration of the solution being delivered.

As shown in FIGS. 2 and 6, the device 100 includes the injector handle 138 for gripping by the operator. The injector handle 138 supports the housing 134 at a proximal end 160 of the housing 134. The injector handle 138 may include a cutout 162 sized and shaped to receive the proximal end 160 of the housing 134. For example, the cutout 162 may be configured to receive a flared end, such as the end of a syringe. The injector handle 138 further includes an opening 168 that is aligned with the proximal opening 152 of the housing 134 so that the plunger advancer member 143 may be received through the openings 168 and 152 and be connected to the plunger 142 inserted into the chamber 136 of the housing 134. The injector handle 138 may be held in one hand by the operator while the other hand rotates the plunger handle 144 to distally advance the plunger member 143. Depending on the type of threads or grooves present on the plunger member 143, the plunger handle 144 may be turned clockwise or counter-clockwise to distally advance the plunger member 143 and the plunger 142.

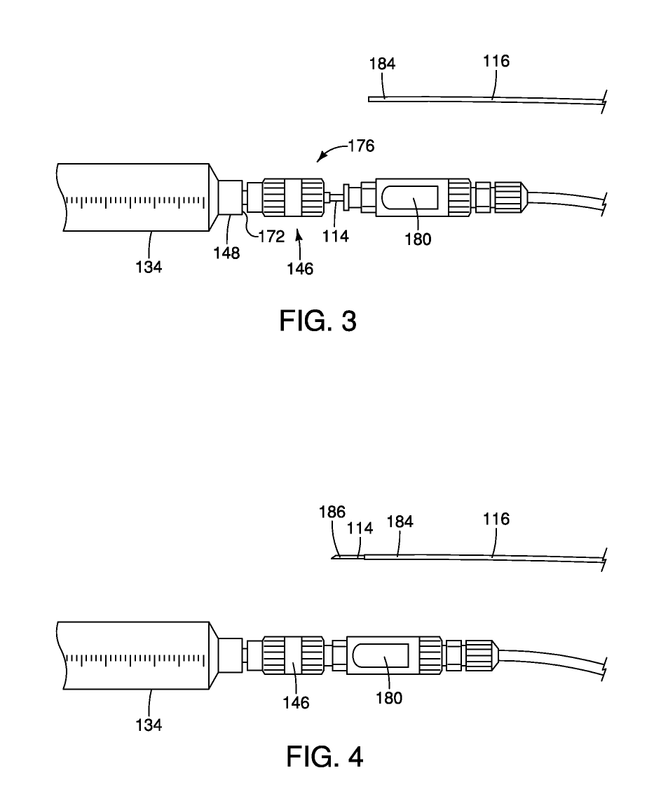

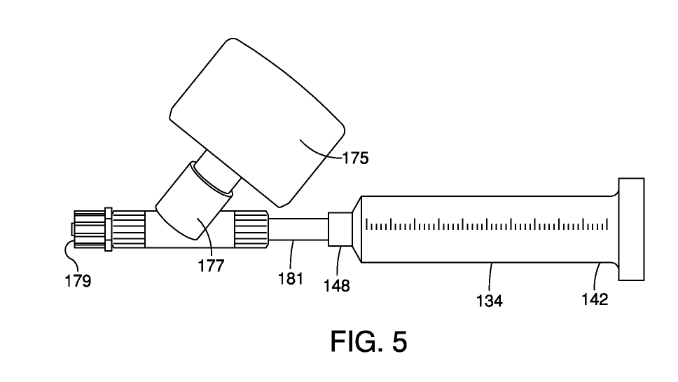

As shown in FIG. 3, the housing 134 also includes an outlet 172 at the distal end portion 148 of the housing 134 for delivering the injectable solution from the chamber 136 to the inner shaft 114. The outlet 172 may include a Luer fitting 174 for connecting with the connector 146 connected to a proximal end 176 of the inner shaft 114. An additional connector 180 may be provided on the outer catheter 116 for removably connecting with the connector 146. The connector 180 may also be tightened against the inner shaft 114 to hold the outer catheter 116 in position relative to the inner shaft 114. As shown in FIG. 3, a distal end 184 of the outer catheter 116 may be positioned to cover a distal end 186 the inner shaft 114, for example during delivery to the tissue treatment site 110. (Compare with FIG. 4.)

The connector 180 may also be released so that the outer catheter 116 is movably positionable relative to the inner shaft 114 to expose a distal end 186 of the inner shaft 114 as shown in FIG. 4. The connector 180 may be connected to the connector 146 and the distal end 186 of the inner shaft 114 distally extended from the outer catheter 116 to a maximum length. The outer catheter 116 is movably positionable so that any length of the distal end 186 of the inner shaft 114 may be exposed between a maximum length and no exposure. By way of non-limiting example, 0-15 mm of the distal end 186 of the inner shaft 114 may be distally extended from the outer catheter 116. Preferably, 7-12 mm of the distal end 186 of the inner shaft may be distally extended from the outer catheter 116. The length of the distal end 186 of the inner shaft exposed will depend on the depth of the tissue to be penetrated. The distal end of the inner shaft 114 may be pointed, beveled, blunt or any shape suitable for insertion of the distal end 186 through the tissue layer 120. In some embodiments, the inner shaft 114 may provided as a needle, such as a 19, 21, 22, 23 or 25 gauge needle, although any size inner shaft 114 may be used. In some embodiments, the inner shaft 114 and outer catheter 116 may be delivered to the tissue treatment site 110 through the working channel of an endoscope and the size of the inner shaft 114 and the outer catheter 116 will depend on the size of the working channel. For example, the inner shaft 114 may be provided as a 19 gauge needle that is extendable through the working channel of an endoscope as shown in FIGS. 3, 4 and 12. The inner shaft 114 includes a uniform inner diameter from the proximal end 176 to the distal end 186. The inner shaft 114 having a gauge of 19 or greater allows for easier navigation through a lumen and provides a uniform conduit for the viscous fluid.

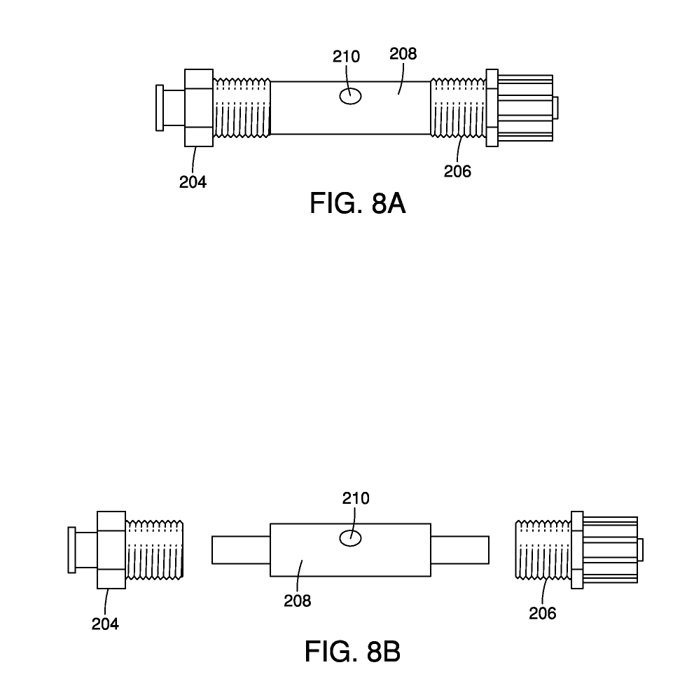

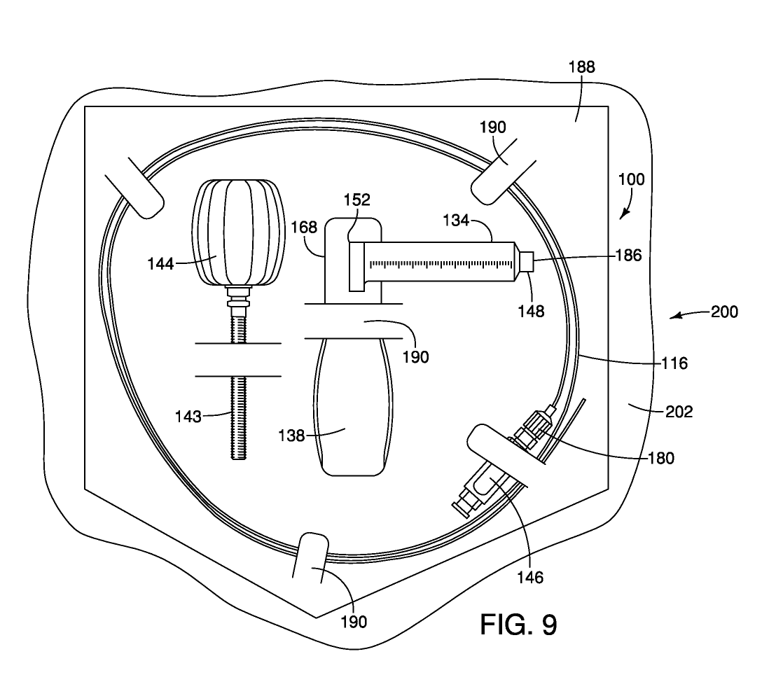

An embodiment of the delivery device 100 is shown in FIG. 7 including a t-shaped fitting 202 connecting the pressure gauge 175 to the housing 134 and the connector 146 of the inner shaft 114. A first adaptor 204 is connected to the connector 146 and a second adaptor 206 is connected to the housing 134. As shown in FIGS. 8A and 8B, the second adaptor 206 may be a female Luer lock adaptor and the first adaptor 204 may be a male Luer lock adaptor. As shown in FIGS. 8A and 8B, a tube 208 may be provided that extends between the adaptors 204, 206. The tube 208 extends within the t-shaped fitting 202 and facilitates reduction of lost solution volume within the fitting 202 as the pressure of the solution is measured by the gauge 175. An opening 210 is provided in the tube 208 to allow the solution to reach the gauge 175. In some embodiments, the fitting 202 may be formed from stainless steel or any suitable material able to withstand the pressure flowing through the fitting 202. The adaptors 204, 206 and the tube 208 may be formed from nylon or any suitable material able to withstand the pressure flowing therethrough.

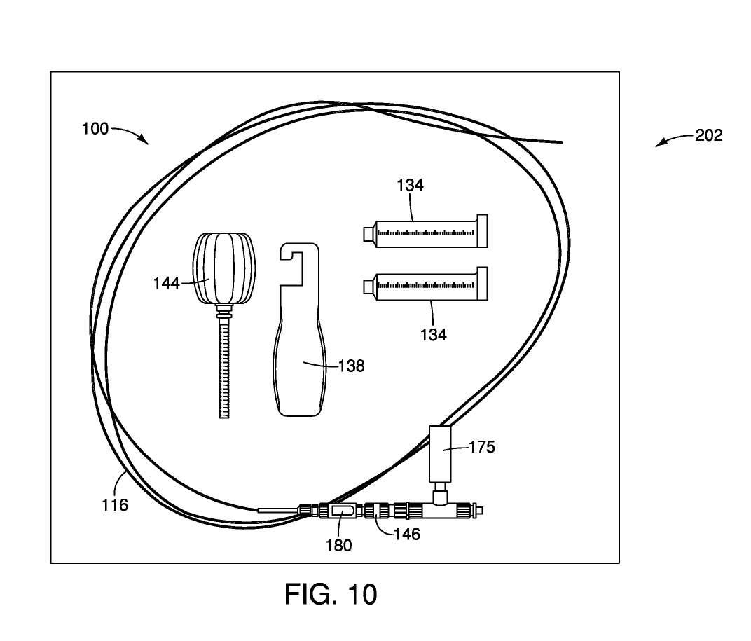

The delivery device 100 may be provided in a kit 200 as shown in FIG. 9. In this embodiment, the kit 200 includes the housing 134, the injector handle 138, the plunger 142, the plunger advancer member 143, the plunger handle 144 and the inner shaft 114 and outer catheter 116. The inner shaft 114 is positioned within the outer catheter 116 and secured by the connector 180 so that the distal end 186 of the inner shaft 114 is completely covered by the outer catheter 116. The plunger advancer member 143 and plunger handle 144 may be provided connected together and separate from the housing 134 and the plunger 142. The injector handle 138 may be provided within the kit pre-connected to the housing 134. The housing 134 may be pre-filled with the injectable solution premixed and ready to be injected directly from the housing 134. The opening 168 in the injector handle 138 and/or the opening 152 in the housing 134 may be protected with a removable seal, a frangible seal or the plunger 142 alone or the like so that the injectable solution remains sterile and contained within the housing 134. The distal end 148 may be provided with a cap 186 to secure closure of the distal end 148 to maintain the sterility and containment of the injectable solution. The components of the kit 200 may be secured to a support member 188 using a plurality of tabs 190 to hold each of the components to the support member 88. The kit 200 may be enclosed within an outer package 202 and the outer package 202 may provide a sterile enclosure for the kit 200.

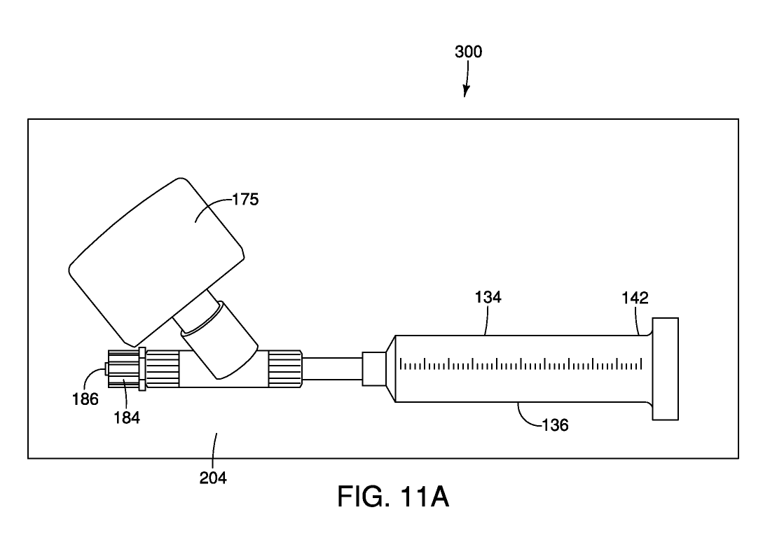

In some embodiments, a kit 202 may also be provided with a plurality of housings 134 as shown in FIG. 10. The plurality of housings 134 may include different concentrations of the injectable solution or each housing 134 having the same concentration, for example, for treatment of multiple tissue lesions in the same patient or treatment of a single large lesion. The volume of the injectable solution provided in housing 134 of the kit 202 may be any volume suitable for a patient treatment. By way of non-limiting example, the suitable volume provided in the housing 134 may be about 1 cc to 50 cc. However, greater or smaller volumes may be provided depending on the size of the lesion(s) and the number of treatments being provided.

As shown in FIG. 10, the kit 202 may be provided with the housing(s) 134 provided separately and prefilled with the injectable solution. Both ends of the housing 134 are sealed to maintain sterility of the injectable solution within the kit 202. The pressure gauge 175 may be provided connected to the connectors 146, 180 and the inner shaft 114 and the outer catheter 116. The handle 138 may be provided separately. The plunger advancer member 143 and plunger handle 144 may be connected together and provided separate from the other components of the kit 202.

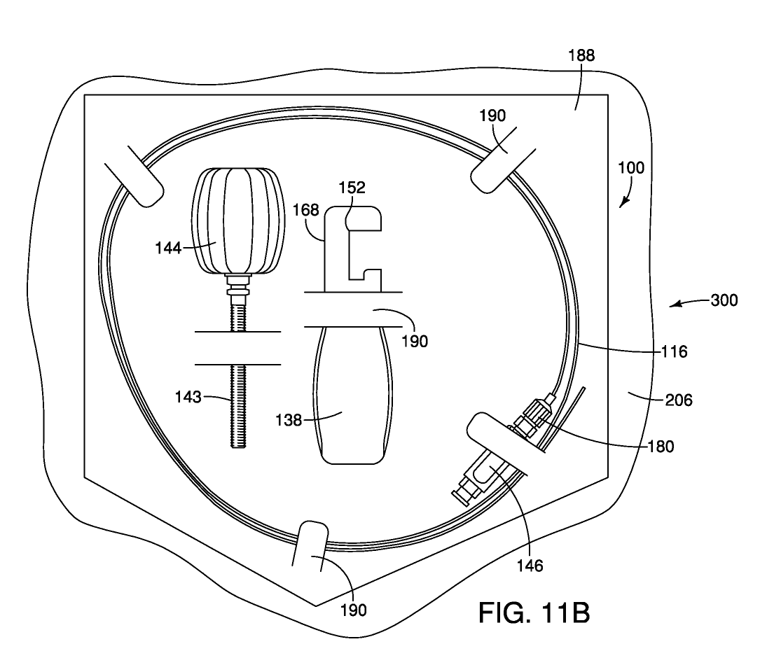

As shown in FIGS. 11A and 11B, a kit 300 may be provided with the housing 134 prefilled with the injectable solution provided within the chamber 136 may be packaged separately from the other components. The pressure gauge 175 when included with the delivery system 100 may also be provided operably connected to the housing 134. The separately packaged housing 134 and the solution therein may be sterilized, for example, using gamma irradiation, and packaged in a package 204. The plunger 142 may provide the seal at the proximal end of the chamber 136 or an additional seal as described above may be included. The distal end 148 may be provided with a cap 186 to secure closure of the distal end 148 to maintain the sterility and containment of the injectable solution. As shown in FIG. 8B, the handle 138, the plunger member 143 connected to the plunger handle 144 and the inner shaft 114 is positioned within the outer catheter and secured by the connector 180 so that the distal end 186 of the inner shaft 114 is completely covered by the outer catheter 116 may be provided in a second package 206. The two packages 204 and 206 may be provided together in the kit 300. Alternatively, the kit 300 may include the first package 204 and the other components provided separately.

An injectable solution suitable for use with the delivery device 100 and suitable for being provided within the housing 134 of the kit 200 is described below. The injectable solution is a pharmaceutically acceptable solution for use in humans and animals that has minimal tissue reactivity. In some embodiments, the injectable solution has a viscosity greater than about 10,000 cP, and in some embodiments, a viscosity greater than about 30,000 cP and greater than about 50,000 cP. The preferred viscosity for the injectable solution is between about 10,000 to 150,000 cP, and in some embodiment the preferred viscosity for the injectable solution is between about 30,000 cP and about 120,0000 cP, although other viscosities may be used. The viscosity of the injectable solution preferably should be high enough to separate the tissue layers. Non-limiting examples of suitable materials for inclusion in the injectable solution include methylcelluloses, such as carboxymethyl cellulose (CMC) and hydroxypropyl methylcellulose (HPMC), extracellular matrix proteins, elastin, collagen, gelatin, fibrin, agarose, and alginate or mixtures thereof. The injectable solution with be described with reference to CMC although one skilled in the art will understand that other suitable materials may also be used to form the injectable solution.

Suitable concentrations of the CMC for the injectable solution include about 1% to 10% CMC (e.g. about 1%, 1,5, 2%, 2.5%, 3%, 3.5%, 4%, 4.5%, 5%, 5.5%, 6%, 6.5%, 7%, 7.5%, 8%, 8.5%, 9%, 9.5%, or 10%). Preferably CMC concentrations range from about 2.5% to 3.5%, and more preferably about 3%. The CMC may be mixed with sterile water, saline or other pharmaceutically acceptable solution to provide a suitable concentration for injection. (CMC may be purchased from Sigma Aldrich, St. Louis, Mo.) The injectable solution may also include additional components, including, but not limited to dyes, such as food coloring, methylene blue or carbon black, and hemostasis regulators, such as vasoconstrictors, for example, epinephrine.

In operation, the CMC is premixed with a pharmaceutically acceptable solution at the manufacturer to the desired concentration for the injectable solution. The CMC injectable solution is loaded into the housing 134 at the manufacturer and the housing 134 is sealed under sterile conditions to maintain the sterility of the CMC injectable solution for delivery to the patient. The remaining components of the kit 200 are assembled together on the support member 88 and packaged for delivery to the physician.

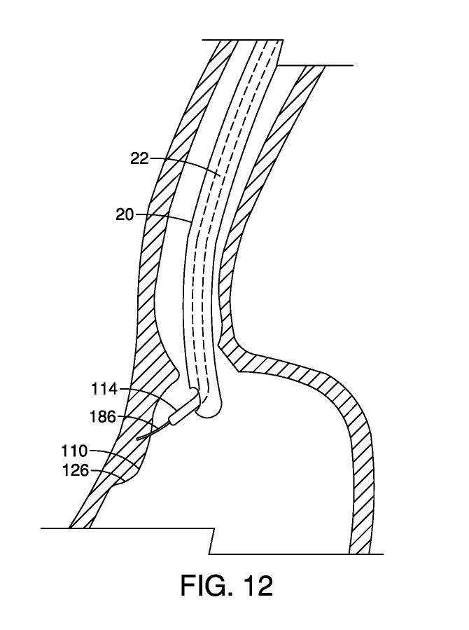

The ESD procedure is described herein with reference to removal of a gastric lesion as shown in FIG. 12, however, the procedure may be performed anywhere in the body having lesions formed in a layer of tissue. The physician may access the tissue treatment site using an endoscope 20 having a visualization port for advancement through a bodily lumen to the site using a wire guide. The distal portion 112 of the delivery device 100 may be advanced to the tissue treatment site 110 through a working channel 22 of the endoscope 20. The distal end 186 of the inner shaft 114 is covered by the outer catheter 116 during advancement to the tissue site 110. The distal end 186 of the inner shaft 114 is extended distal to the outer catheter 116 and advanced into the first layer of tissue 120 at the treatment site 110. The length of the distal end of the inner shaft 114 is extended will depend on several factors, including, but not limited to, the size of the lesion and the depth of the tissue wall that is to be elevated by the injectable solution. The depth and extension of the inner shaft 114 will be determined and monitored by the physician. In some embodiments, the distal end 186 of the inner shaft 114 may be extended about 5-15 mm beyond the outer catheter.

The physician can monitor the depth of the injection required using the visualization port of the endoscope. An injection of saline or other pharmaceutically acceptable solution may be used to initiate the formation of the tissue pocket 126. The CMC injectable solution is injected into the same injection site through the inner catheter 114 in an amount sufficient to create the tissue pocket 126 for a time sufficient for the procedure. The CMC injectable solution is injected under sufficient pressure and with a sufficient volume and viscosity to break the cellular attachments between the first layer 120 and the second layer 122 at the tissue treatment site 110. A dye may be included with the CMC injectable solution to help the physician visualize the elevated portion of the tissue. The amount of CMC injectable solution injected to form the tissue pocket 126 is determined by the physician. Once the tissue pocket is formed, the inner shaft 114 is removed and an electrocautery device or snare is inserted into the working channel and advanced distally to the treatment site 110 and the diseased tissue removed.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

1. A kit for delivering an injectable solution to a tissue treatment site, the kit comprising;
a housing having a chamber therein, a proximal portion and a distal portion;
an injectable solution provided in the chamber, the injectable solution having a viscosity greater than about 10,000 cP;
a plunger movably positionable within the proximal portion of the chamber, the plunger providing a seal at the proximal end portion of the housing to prevent the injectable solution from flowing out of the proximal end portion; and
a pressure gauge operably connectable to the housing.

2. The kit of claim 1 further comprising:
a handle connectable to the housing;
a plunger advancing member having a plunger handle connected thereto, the plunger advancing member provided separate from the housing and having a distal portion configured for operably connecting with the proximal portion of the housing; and
an inner shaft, the inner shaft provided separate from the housing and having a proximal end portion configured for operably connecting with the distal portion of the housing for receiving the injectable solution therethrough and a distal end of the inner shaft configured for insertion in to the tissue treatment site.

3. The kit of claim 1, wherein the injectable solution has a viscosity greater than about 30,000 cP.

4. The kit of claim 2, wherein the inner shaft comprises a 19 gauge needle.

5. The kit of claim 1, wherein the injectable solution comprises carboxymethyl cellulose.

6. The kit of claim 4, wherein the carboxymethyl cellulose concentration is about 2.5% to about 3.5%.

7. The kit of claim 1, wherein the injectable solution comprises a dye.

8. The kit of claim 1, wherein the device further includes a fitting operably connected to the pressure gauge, a first adaptor and a second adaptor connected to the fitting, and a tube extending within a lumen of the fitting and extending between the first adaptor and the second adaptor.

9. The kit of claim 2, further comprising an outer catheter provided separate from the housing and movable in relation to the inner shaft to expose a portion of the distal end of the inner shaft for insertion of the distal end into the tissue treatment site.

10. The kit of claim 9, wherein the outer catheter is movable in relation to the inner catheter.

11. The kit of claim 2, further comprising an outer packaging for enclosing the housing, the handle, the plunger and the inner shaft.

12. The kit of claim 2, wherein the plunger advancing member comprises threads for distally advancing the plunger into the chamber.

13. The kit of claim 1, wherein the chamber is a sealed chamber for maintaining the sterility of the injectable solution.

14. A kit for delivering an injectable solution to a tissue treatment site, the kit comprising;
a housing having a chamber therein, a proximal portion and a distal portion;
an injectable solution provided in the chamber, the injectable solution having a viscosity greater than about 10,000 cP;
a plunger movably positioned within the proximal portion of the chamber, the plunger providing a seal at the proximal end portion of the housing to prevent the injectable solution from flowing out of the proximal end portion;
a handle connectable to the housing;
a plunger advancing member having a plunger handle connected thereto, the plunger advancing member provided separate from the housing and having a distal portion configured for operably connecting with the proximal portion of the housing; and
an inner shaft, the inner shaft provided separate from the housing and having a proximal end portion configured for operably connecting with the distal portion of the housing for receiving the injectable solution therethrough and a distal end of the inner shaft configured for insertion in to the tissue treatment site.

15. The kit of claim 14 wherein, the kit further comprising a pressure gauge operably connected to the housing.

16. A method of elevating a first tissue layer away from a second tissue layer, the method comprising:
connecting an inner shaft to a distal portion of a housing having a chamber therein;
connecting a plunger to a proximal portion of the housing;
advancing a distal end of the inner shaft to the first tissue layer and inserting the distal end into the first tissue layer;
distally advancing the plunger to advance an injectable solution having a viscosity greater than about 10,000 cP from the chamber through the inner shaft and into the tissue; and
injecting the solution into the first layer and elevating the first tissue layer away from the second tissue layer.

17. The method of claim 16, further comprising providing an outer catheter movably positioned over the inner shaft and advancing the outer catheter together with the inner shaft to the first tissue layer.

18. The method of claim 17, comprising extending the inner shaft distally relative to the outer catheter for insertion of the inner shaft into the first tissue layer.

19. The method of claim 17, comprising advancing the inner shaft through a working channel of an endoscope.

20. The method of claim 17, comprising providing the injectable solution comprising carboxymethyl cellulose.
